(19) United States
(12) Patent Application Publication
(10) Pub. No.: US 2022/0084622 A1
(43) Pub. Date: Mar. 17, 2022

(54) SYSTEM FOR SIMULATING MOLECULAR INTERACTIONS INVOLVED IN INFLAMMATION

(71) Applicant: Biologische Heilmittel Heel GmbH, Baden-Baden (DE)

(72) Inventors: Shailendra Kumar GUPTA, Rostock (DE); Konstantin CESNULEVICIUS, Gernsbach (DE)

(73) Assignee: Biologische Heilmittel Heel GmbH, Baden-Baden (DE)

(21) Appl. No.: 17/422,726

(22) PCT Filed: Jan. 21, 2020
(86) PCT No.: PCT/EP2020/051376

§ 371 Date: Jul. 13, 2021

(30) Foreign Application Priority Data
Jan. 21, 2019 (EP) 19152836.3

(51) Int. Cl. G16B 5/10

(52) U.S. Cl. CPC G16B 5/10

(57) ABSTRACT

The present invention relates to the filed of simulation of molecular interactions for assessing diseases and disease therapies. In particular, it relates to a system for simulating molecular interactions involved in inflammation in a subject, said system comprising a processing unit comprising a database comprising a plurality of datasets each comprising at least an identifier for a molecule suspected to be involved in the pathological process, data on molecular interactions of the said molecule with one or more other molecule, and at least one data characteristic that is allocated to the dataset, wherein each dataset has at least one relation with another dataset in the database based on molecular interactions, wherein the datasets are grouped into data compartments comprising datasets having identical data characteristics, and wherein the data characteristics are indicative for the biological function of a molecule in inflammation, and an computer program-based algorithm implemented in the processing unit which generates a network map based on the plurality of datasets in the database and which allows for identifying nodes within network map based on predefined parameters, and a visualization unit which allows for determination of the molecular interactions in the identified nodes. Moreover, the present invention contemplates a method for simulating molecular interactions involved in inflammation in a subject as well as the use of the system of the invention for simulating molecular interactions involved in inflammation.

The present invention relates to the filed of simulation of molecular interactions for assessing diseases and disease therapies. In particular, it relates to a system for simulating molecular interactions involved in inflammation in a subject, said system comprising a processing unit comprising a database comprising a plurality of datasets each comprising at least an identifier for a molecule suspected to be involved in the pathological process, data on molecular interactions of the said molecule with one or more other molecule, and at least one data characteristic that is allocated to the dataset, wherein each dataset has at least one relation with another dataset in the database based on molecular interactions, wherein the datasets are grouped into data compartments comprising datasets having identical data characteristics, and wherein the data characteristics are indicative for the biological function of a molecule in inflammation, and an computer program-based algorithm implemented in the processing unit which generates a network map based on the plurality of datasets in the database and which allows for identifying nodes within network map based on predefined parameters, and a visualization unit which allows for determination of the molecular interactions in the identified nodes. Moreover, the present invention contemplates a method for simulating molecular interactions involved in inflammation in a subject as well as the use of the system of the invention for simulating molecular interactions involved in inflammation.

Inflammation is a response of the body to harmful stimuli such as pathogens, damage or physical and chemical irritants resulting in cellular injury. It is a protective measure involving cells of the immune system, and blood vessels serving to eliminate the cause of cellular injury, to remove necrotic cells and tissues damaged, and initiate tissue repair.

Inflammation is a complex biological process which can be divided into acute inflammatory processes and chronic inflammatory processes. Cardinal signs of inflammation include pain, increased body temperature, redness, swelling, and functional impairments.

Acute inflammation, typically, involves resident immune cells present in the affected tissue. These cells exhibit surface receptors known as pattern recognition receptors (PRRs), which recognize two subclasses of molecules: pathogen-associated molecular pattern molecules (PAMPs) and damage-associated molecular pattern molecules (DAMPs). PAMPs are pathogen-specific molecules while DAMPs are molecules being associated with host-related injury and cell damage.

At the onset of inflammation the PRRs recognize a PAMP or DAMP and induce inflammatory mediators responsible for the clinical signs of inflammation as well as several cellular and extra-cellular biochemical cascades propagating the inflammatory response (see, e.g., Netea 2017).

It will be understood that drugs involved in treating various aspects of inflammation may act on various levels of regulation in the complex molecular pathways involved in the different phases of inflammation. It would, thus, be useful to investigate the molecular processes involved in inflammation in a simulation in order to predict drug effects and therapy outcome.

Recently, in silico models have been reported which are able to simulate complex molecular interactions in genomic and transcriptomic analyses (Wolkenhauer 2002, Steffen 2017). Moreover, regulators networks of genes and disease simulations have been established for various types of cancer (see, Sadeghi 2016, Dryer 2018, and Khan 2018).

However, in simulations for inflammation that may facilitate in silico analyses of inflammatory processes and drug development are not available yet but would be highly desired.

The technical problem underlying the present invention is, thus, the provision of means and methods for complying with the aforementioned needs. The technical problem is solved by the embodiments characterized in the claims and herein below.

Therefore, the present invention relates to a system for simulating molecular interactions involved in inflammation in a subject, said system comprising
(I) a processing unit comprising
   (a) a database comprising a plurality of datasets each comprising
      (i) at least an identifier for a molecule suspected to be involved in the pathological process,
      (ii) data on molecular interactions of the said molecule with one or more other molecule, and
      (iii) at least one data characteristic that is allocated to the dataset,
      wherein each dataset has at least one relation with another dataset in the database based on molecular interactions;
      wherein the datasets are grouped into data compartments comprising datasets having identical data characteristics; and
      wherein the data characteristics are indicative for the biological function of a molecule in inflammation;
      and
   (b) an computer program-based algorithm implemented in the processing unit which generates a network map based on the plurality of datasets in the database and which allows for identifying nodes within network map based on predefined parameters; and
(II) a visualization unit which allows for determination of the molecular interactions involved in inflammation in the identified nodes.

The term “system” as used herein refers to any assembly comprising (I) the processing unit and (II) the visualization unit referred to above. It will be understood that each of said units may also consist of several separate devices or sub-units. For example, the processing unit may comprise a storage device for the database comprising a plurality of datasets referred to above and a processor having implemented the computer program-based algorithm which generates a network map based on the plurality of datasets in the database and which allows for identifying nodes within network map based on predefined parameters. Further, it may comprise a device for data input such as a device for automatic data input, e.g., a reader device or a receiving device for data from other data processing devices or the internet, or a device for manual data input. The system shall, preferably, also comprise components for data transmission between the individual units, subunits and/or devices. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. The units and other components of the system may be arranged in physical proximity or may be physically separated from each other.

The term “simulating” as used herein refers to establishing virtual model of the molecular interactions involved in inflammation in the system according to the invention. Such a simulation, preferably, is a dynamic simulation, i.e. it takes into account changes on the interactions over time and in response to simulated external influences. Moreover, the simulation may be a deterministic simulation excluding statistical events or may be include such events.

The term “molecular interactions involved in inflammation” as referred to herein physiological and pathophysiological interactions between biological molecules known to be present in the subject and known to be involved in inflammation. Inflammation as used herein, typically, refers to a pathophysiological response of a subject against harmful stimulation by, e.g., pathogens, cellular damage or irritants. Inflammation may be acute or chronic inflammation. Preferably, inflammation referred to in accordance with the present invention is chronic inflammation. Typical signs of inflammation are heat, pain, redness or other skin reactions, swelling and impaired tissue function. Inflammation, typically, starts when tissue-resident cells of the innate immune system detect infection or damage within the tissue. Secretion of chemical signals such as chemokines and cytokines lead to the recruitment of circulating neutrophils to the site of damage or infection. Lipid mediator class switching occurs as neutrophils congregate in pus or purulent exudates. Lipoxins (LXs) stimulate non-phlogistic monocyte recruitment. LXs, resolvins (Rvs), and other specialized pro-resolving mediators (SPMs) are produced in pus to limit or stop further neutrophil tissue infiltration. SPMs, Rvs, maresins (MaRs), and protectins, each stimulate efferocytosis of apoptotic neutrophils and cellular debris by macrophages. Resolving macrophages and apoptotic neutrophils also produce SPMs. Edema also brings circulating n-3 polyunsaturated fatty acids (PUFAs) into exudates for temporal conversion to SPMs by exudate cells. MaRs and specific Rvs enhance wound healing and tissue regeneration. Afterwards, tissue homeostasis can be restored. There are various inflammatory diseases where the resolution of inflammation is apparently impaired.

The molecules that participate in the molecular interactions to be simulated in accordance with the present invention can be determined by several techniques. Typically, pre-defined molecules known to be involved in inflammation or molecules known to be associated with pre-defined inflammatory indications are used as a first set of molecules (also hereinafter called “seed molecules”) to be included in the database of the system of the invention.

In the case of inflammation, the seed molecules in a first step are, preferably, from a damage associated molecular pattern (DAMP) and/or from a pathogen associated molecular pattern (PAMP). The molecules may be proteins, peptides, nucleic acids, such as RNAs and DNAs, and/or the biological molecules such as lipids or metabolites. The molecules involved in molecular interaction in inflammation and, in particular, the molecules of DAMP and/or PAMP are, preferably, identified by evaluating scientific data. Alternatively, the molecules may also be identified by evaluating studies aiming at identifying such molecules. Typically, available scientific data in, e.g., scientific databases such as PubMed or OMIM may be evaluated by automated text mining algorithms which identify molecules suspected to be involved in inflammation, i.e. by a literature mining process. Suitable algorithms for identifying molecules involved in inflammation may identify key words and/or key parameters in the scientific data that are associated with a role in inflammation. A preferred method for identifying molecules that are involved in the molecular interactions in inflammation is the method described by Khan et al. (Khan, 2018). Further details on the identification of such seed molecules are described in the accompanying Examples, below.

DAMP associated molecules are, preferably, selected in a first step from the group consisting of: high-mobility group box 1, Glycosaminoglycan hyaluronan, Heparan sulphate, Uric Acid, interleukin (IL)-1α, interleukin (IL)-1β, interleukin 16, interleukin 18, fibrobast growth factor, Galectin-3, Galectin-1, endothelial monocyte-activating polypeptide-II, Macrophage migration inhibitory factor, NLR Family Pyrin Domain Containing 3, PYD And CARD Domain Containing, cysteine-aspartic acid protease 1, Cross-linked dimer of ribosomal protein S19, Lysophosphatidylcholine, Intracellular Membrane-Associated Calcium-Independent Phospholipase A2 Beta, Tyrosyl-TRNA Synthetase, Protein S100-A8, Protein S100 A-9, and Calreticulin.

PAMP associated molecules are, preferably, selected in a first step from the group consisting of Cytoplasmic DNA, Bacterial Flagelli, Bacterial Type III Secretion Systems, Bacillus Anthracis Lethal Toxin, ATP, Marine toxin maitotoxin, Crystals (Urate, Calcium Pyrophosphate Dihydrate, Silica, Asbestos), Diaminopimelic Acid, Muramyl Dipeptide, Triacyl Lipopeptides, Peptidoglycan, Lipoarabinomannan, Envelope Glycoproteins, Phospholipomannan, Porins, Diacyl Lipopeptides, Lipoteichoic acid, dsRNA, Lipopolysaccharide, Mannan, Flagellin, ssRNA and their recognizing recptors. The recognizing receptors are AIM2, NLRC4, NLRP1, NLRP3, NOD1, TLR1, TKR2, TLR6, TLR3, RIG1, DDX58, IFIH1, EIF2AK2, TLR4, TLR5, TLR7, and TLR8.

In yet a further step, additional seed molecules can be identified by evaluating scientific data referring to acute inflammatory indications by a literature mining process which aims at identifying molecules associated with the said acute inflammatory indications. Typically, acute inflammatory clinical indications are selected from the group consisting of: Gonococcal Arthritis, Viral Arthritis, Tendonitis, Bursitis, Acute Spinal Disc Injury, Muscle Injury/Muscle Tear, Soft Tissue Injuries, Epiphyseal Injuries, Overuse Injuries, Ankle Sprain, Talofibular Ligament Injury, Hip Tendonitis and Bursitis, Contusions, Medial Collateral Knee Ligament Injury, Posterior Cruciate Ligament Injury, Iliotibial Band Syndrome, Acromioclavicular Joint Injury, Rotator Cuff Injury, Supraspinatus Tendonitis, Cervical Spine Sprain/Strain Injuries, Sacroiliac Joint Injury, Bicipital Tendonitis, Lateral Epicondylitis, Medial Epicondylitis, Cervical Sprain and Strain, Achilles Tendon Injuries, Calcaneal Bursitis, Hamstring Strain or Biceps Rupture. More preferably, at least the following acute clinical indications are evaluated for additional seed molecules involved in inflammation: Bursitis, Subacrominal Bursitis, Olecranon Bursitis, Tendinitis, Tenosynovitis, Epicondylitis, De Quervain disease and acute Arthritis. The literature mining process, typically, includes the automated screening of scientific literature in databases, such as PubMed and/or OMIM, and the screening of disease-gene associated databases such as DisGeNET, DISEASE, KEGG disease) to find the genes associated with the clinical indication by text mining algorithms. Further details on the identification of such seed molecules are described in the accompanying Examples, below.

The additional seed molecules, preferably, comprise IL1b, IL6, COX-2, CXCL12, VEGF-A, KDR, TP53, SIRT1, HLA-b, HLA-C, TNF, PRG4, COL5A1, ESR2, GPI, and RS19.

For the aforementioned seed molecules, a database is established in the system of the invention. Said database shall comprise a plurality of datasets for each seed molecule, said datasets each comprising:

(i) At least an identifier for a molecule suspected to be involved in the pathological process. Typically, a scientific designation or name for the molecule can be used. Alternatively, a unique number or unique arbitrary identifier can be used which, in a subsequent step, can be unambiguously allocated to the scientific designation or name for the molecule. More preferably, for each seed molecule, typically, its specific UNIPROT-, HGNC-, RefSeq-, Ensemble-, PubMed- and NCBI-ID, CHEBI-ID as well as common aliases and the full name of the encoding Protein will be included.

(ii) Data on molecular interactions of the said molecule with one or more other molecule. Typically, molecular interaction partners or data pertaining to molecular regulators of the seed molecules shall be included. More preferably, such data may comprise information on source and target molecules, regulators such as enzymes, expression control molecules such as transcription factors or regulatory RNAs, etc. Moreover, the data may also comprise information on the type of modification (e.g. catalysis, phosphorylation), state of the molecules (e.g. phosphorylated), type of the molecules (proteins, miRNA, complexes, DNA, simple molecules), and/or type of the interaction (positive, negative). Such information can be automatically identified by mining algorithm such as BisoGenet app available on Cytoscape4.0.

(iii) At least one data characteristic that is allocated to the dataset. The data characteristic shall be indicative for the biological function of the seed molecule such that the molecule can based on said information be allocated into a functional compartment of the inflammation process. The functional compartment of the inflammation process is, preferably, selected from the group consisting of: regulation of hemopoiesis, initiation of innate immune response, pattern recognition receptor signaling, regulation of adaptive immune response, T-cell mediated immune response, cytokine production, regulation of immunoglobuline secretion, mast cell degranulation, T-cell selection, Immune cell differentiation, lymphocyte differentiation, myeloid differentiation, regulation of lymphocyte proliferation, regulation of B-cell proliferation and regulation of T-cell proliferation.

The aforementioned information to be included into the datasets for the seed molecules can be also identified by evaluating scientific data, typically, available scientific data in databases for interaction partners or regulators. Mainly experimentally validated molecular targets from DIP, BioGrid, HPRD, IntAct, MINT, BIND and String databases can be considered. Furthermore, several experimentally validated regulatory layers, which may include miRNAs from mirBase, miRTarBase, TriplexRNA; transcription factors from TRNSFAC, TRRUST and HTRIdb; lnc RNAs from lncRInter, EVLncRNAs, lncRNADisease databases can be applied. It is already evident that fatty acid metabolism play an important role in the biosynthesis of specialized pro-resolving molecules (including resolvins, protectins and maresins). Therefore, information about the biosynthesis of these inflammation resolution mediators along with all the associated enzymes may also be collected and included from Reactome database and published literature. Further details on the identification of such seed molecules are described in the accompanying Examples, below.

The aforementioned datasets of the seed molecules are structured in the database of the system of the invention in that each dataset has at least one relation with another dataset in the database based on molecular interactions, datasets are grouped into data compartments comprising datasets having identical data characteristics based on the data characteristics of the datasets being indicative for the biological function of a seed molecule in inflammation, i.e. the seed molecule datasets are grouped into functional compartments of the inflammation process. Suitable algorithms for structuring the data and for building up the functional compartments of the inflammation process comprise the CytoScape plugin ClueGO, which utilizes the GeneOntology (GO) database. The results can be adjusted manually by defining parameters, e.g. the range of tree levels of the term in the GO-Hierarchy, the p-value for the association or the amount of genes per modules. To focus on immunology related processes, the GO database “immunological_process” can be, preferably, used in the first run of modularization. In a second run, a more general database “biological_process” may be applied. Further details on the identification of such seed molecules are described in the accompanying Examples, below.

The interactions to be simulated according to the present invention may be associated with biological functions of the molecules in the inflammation process. Such biological functions may be selected from the group consisting of: Mast Cell degranulation, Macrophage Differentiation, Myeloid Cell Differentiation, Lymphocyte Differentiation, Immune Cell Differentiation, Regulation of Hemopoiesis, Initiation of Innate Immune Response, Pattern recognition Receptor Signaling Pathways, Cytokine Production, Regulation of Adaptive Immune Responses, T-cell Mediated Immune Response, T cell Selection, Regulation of B cell Proliferation, Regulation of Immunoglobulin Secretion, Regulation of T cell Proliferation, Regulation of Lymphocyte Proliferation, Inflammation Resolution, Gene Expression Regulation, Protein Modification, Blood Vessel Development, Immune Response, Hemopoiesis, Neuronal Development, and Protein Transport.

The seed molecules are allocated to by the database structure to the aforementioned biological functions resulting in a functional compartment structure as described before. Moreover, it will be understood the additional information comprised by the seed molecule datasets on interaction partners and regulators as well as other information is also allocated to said functional compartments of inflammation.

The term “processing unit” as referred to herein refers to a data processing device on which the computer program-based algorithm according to the inventions runs and which is functionally connected to the database of the system of the invention. Thus, the processing unit in the system of the invention is configured by tangibly embedding the computer program-based algorithm for carrying out the method of the invention on said system. The processing device may comprise at least one integrated circuit configured for performing logical operations. Typically, the processor may also comprise at least one application-specific integrated circuit (ASIC) and/or at least one field-programmable gate array (FPGA). How to functionally connect the database which may be stored on a temporary or permanent physically integrated data storage device or which may be accessible by remote connection is well known to those skilled in the art.

The term “computer program-based algorithm” as used herein to computer-readable rules that implement an algorithm which is capable of generating a network map of the seed molecules and accompanying information based on the plurality of datasets in the database. Said network map of the seed molecules and the interaction partners and regulators thereof allows for identifying certain nodes within the network map based on predefined parameters. The aforementioned nodes are integration points within the network. A node is characterized in that a seed molecule interacts with more than two molecules. Preferably, the more than two molecules are more than two other seed molecules. However, the more than two molecules may also be molecules included into the database as accompanying information, i.e. interaction partners or regulators allocated to a given seed molecule. The algorithm to be applied, typically, comprises rules for gene prioritization, determining node degree, determining betweennecess centrality, motif identification, in particular, identification of feedback or feedforward loops, and/or determining association with inflammation.

The term “network map” as used herein refers to a visualized network of the seed molecules as represented by the datasets in the database of the system of the invention including all or part of the accompanying information on interaction partners and regulators. Typically, a network map as referred to herein may be constructed by computer-based algorithms such as those implemented in the CellDesigner and/or Cytoscape software. Further details are also described in the prior art (Khan 2018). In accordance with the present invention, additional step by step procedures were developed in order to arrange network components and interactions for better visualization. In a first step, seed molecules and other components in a module are organized based on their interactions with other modules (i.e. inter modular connections) in a way that most of the interacting arrows should not visually cross each other when being graphically visualized. In a subsequent step, the network components are ordered based on their interactions within a module as well as their interactions with other modules of the map. In a further step, other readability issues for components such as proteins and complexes that have many inter- and intra-modular connections can be resolved. Details are found in the Figures and accompanying Examples below.

Preferably, Boolean modeling formalism may be used to simulate the dynamical system behavior of the network for different phenotypes over different time points, such as disease phenotypes and in accordance with the present invention inflammation. Boolean models do not require detailed quantitative kinetic parameters which makes them suitable for dynamical analysis of large regulatory networks. Boolean functions can be trained and/or calibrated with experimental data to make them context/cell type/disease-phenotype specific. In such models, biological components such as genes, proteins, protein complexes and other species are represented in the nodes associated with discrete state values (0 or 1) and the directed/signed interactions between the nodes are represented by Boolean functions consisting of logical gates specifying how the future state of nodes are determine from the current state of their regulators. Details are found in the accompanying Examples below and are described in the art (see, e.g., in Khan 2018).

The term “visualization unit” as used herein refers to any device which is capable of visually displaying the interactions in the nodes of the network map. Typically, such a visualization unit, thus, comprises a device for visual representation of the network map, e.g., screen or display, or may allow for generating a digital or paper print out of the network map. The visualization unit, typically, comprises a computer program-based algorithm which graphically arranges highly connected nodes close to each other and/or which identifies only nodes for graphical display that are intramodularly connected. Typical screen or display devices may be based on EL, LC, LED, OLED, AMOLED, Plasma or quantum dot technologies. Moreover, the visualization unit shall, preferably, comprise a data processor which allows for creating a visualization of the network model on the device for visual representation of the network map. The data processor is typically run by a suitable algorithm implemented via a computer program code in the data processor. Typically, said algorithm may use commercially available computer programs such as Leaflet.js, JavaScript, jQuery, Inkscape and/or Phyton and others referred to herein below.

The network map may be, preferably, visualized in a browser readable format. Moreover, in light of the comprehensive information contained in the network map, it is preferably envisaged to establish a zoomable format for the map. Typically, the network map may be established as an interactive image. Furthermore, algorithms implemented in the OpenLayer and Google Maps software can be typically applied for browser-based visualization. More specifically, the network map can be divided into three layers. Basic layers are modules in the top level, sub-modules and species in the middle layer and species and interactions in the bottom layer; see also accompanying Examples and Figures for details. To reduce the computational effort and the requirements to the local internet, tiling the image was carried out. Tiling is a technique to cut images into a matrix of smaller images of the same size and store it in a specific folder structure. With this, only the currently viewed parts of the network map can be loaded and displayed. Python scripts can be typically used for tiling and layering to test if the CellDesigner export of our map fits to the postproduction process.

The MINERVA [https://minerva.pages.uni.lu] platform may be, preferably, used to visualize the network map. A local MINERVA instance can be installed and tested with the network map for various security and reliability issues. Advantageously, MINERVA provides the possibility to use OpenLayer as the basic visualization library to enhance the data security in comparison to GoogleMaps. The tiling and layering process is fully automated and basic mouse operations for zooming and panning the MIM are also supported. In addition, the user can select nodes to see the underlying annotations and at the same time connect with various drug and chemical databases directly from MINERVA interface for further network analyses. How to apply these software tools is, in general, well known to those skilled in the art and can be applied without further ado. Details can be also found in the accompanying Examples, below.

Providing regulation direction (e.g. activation, inhibition) to the edges connecting various nodes in the network is a crucial step for analyses and prediction of biomarkers and therapeutic candidates. Thus, confirming and/or including such information into the datasets underlying the network map may be also carried out, in particular, if disease conditions shall be simulated. In order to cross-check and/or allocate regulation directions, the publications associated with the datasets may be manually checked and regulation directions may be confirmed or amended based on the outcome of the check. In addition or as an alternative, system biology resources such as the BioModel databases can be used to assign regulatory directions to the connected components using automated scripts. Many of the databases are using text mining approaches to even highlight experimentally validated interactions with false positive information.

Moreover, in the aforementioned system of the invention, other information may be included. Preferably, such other information as referred to herein may be data from transcriptomics, lipidomics and/or metabolomics investigations. More preferably, transcriptomics data may be used. Such transcriptomics data may be, typically, transcriptomics data obtained from a subject or group of subjects after administration of a drug or transcriptomics data indicating the difference between two groups of subjects or a subject or group of subjects prior and after treatment with a drug. More preferably, these other information and, preferably, transcriptomics data are used to model the datasets in the database. Most preferably, datasets from the database are eliminated if no match is found between the identifier for a molecule in the dataset and the molecule identifier in the other information, preferably, the transcriptomics data. Typically, the transcriptomics data comprise information on the expression level for transcripts for seed molecules, regulators or interaction partners comprised in the network map. The data on expression levels may be parameters reflecting absolute levels or relative changes. Moreover, any mathematical derived values from such parameters may be used, e.g., logarithms. Further details on how transcriptomics data may be included into the network map are described herein below and, in particular, in the accompanying Examples below.

More preferably, the network map which can be generated and used by the system of the invention is created by the following steps:

More specifically, in a first step, seed molecules that participate in the molecular interactions to be simulated are identified by literature mining of scientific literature for DAMP, PAMP and clinical indications of acute inflammation. For those seed molecules, the datasets are provided in the database (see FIG. 2).

Subsequently, a network map is construed by further database and literature mining aiming at identifying interaction partners and/or regulators of the seed molecules. Typically, the following databases may be searched: DIP, BioGrid, HPRD, IntAct, MINT, BIND and/or STRING. Interactions may be validated based on experimental data available in the literature. The resulting relationships of seed molecules among each others and their interaction partners and regulators are compiled to a network map. The seed molecules, interaction partners and regulators are also grouped into functional compartments of biological function in inflammation based on the information comprised in the dataset for each seed molecule (see FIG. 2).

Finally, further information may be integrated into the map and be used for validating regulatory relationships, the resulting network map is visualized and can investigated as described above in detail (FIG. 2).

Advantageously, it has been found in accordance with the studies underlying the present invention that a system for simulating molecular interactions can be provided based on a network map of molecular interactions between pre-defined seed molecules and associated interaction partners and regulators. Such a network map provides a scaffold for dynamical systems analysis of diseases. The molecular interactions in the map represent the physical or causal relationships among the entities which can be translated into mathematical equations to analyze the dynamics of the system over time. Boolean modeling formalism may be used to simulate the dynamical system behavior of the network for different disease phenotypes over different time points. Boolean models do not require detailed quantitative kinetic parameters which makes them suitable for dynamical analysis of large regulatory networks. Boolean functions can be trained and/or calibrated with experimental data to make them context/cell type/disease-phenotype specific. After calibrating the model with experimental data, in silico experiments can be performed to mimic the known behavior of the system for different stimuli or initial conditions. Further, the model will be subjected to various perturbations, where the change in the Boolean states of node(s) will be observed in the context of phenotypic outcomes (e.g., resolution of inflammation). These perturbation experiments can be used to detect critical interactions that can be interesting targets for therapy.

In the following, preferred embodiments of the system of the invention are described in more detail. The explanations and definitions of the terms made above apply mutatis mutandis except as specified otherwise.

In a preferred embodiment of the system of the invention, said inflammation comprises inflammation resolution.

In a further preferred embodiment, said molecule is identified from a damage associated molecular pattern (DAMP) or from a pathogen associated molecular pattern (PAMP). More preferably, said DAMP and/or said PAMP are derived from evaluation of publications in public scientific databases using an automated evaluation algorithm which identifies molecules suspected to be involved in inflammation.

In a preferred embodiment of the system of the invention, said biological function of a molecule in inflammation is selected from the group consisting of: Mast Cell degranulation, Macrophage Differentiation, Myeloid Cell Differentiation, Lymphocyte Differentiation, Immune Cell Differentiation, Regulation of Hemopoiesis, Initiation of Innate Immune Response, Pattern recognition Receptor Signaling Pathways, Cytokine Production, Regulation of Adaptive Immune Responses, T-cell Mediated Immune Response, T cell Selection, Regulation of B cell Proliferation, Regulation of Immunoglobulin Secretion, Regulation of T cell Proliferation, Regulation of Lymphocyte Proliferation, Inflammation Resolution, Gene Expression Regulation, Protein Modification, Blood Vessel Development, Immune Response, Hemopoiesis, Neuronal Development, and Protein Transport.

In a preferred embodiment of the system of the invention, said computer program-based algorithm implemented in the processing unit which generates a network map based on the plurality of datasets in the database and which allows for identifying nodes within network map comprises rules for gene prioritization, determining node degree, determining betweennecess centrality, motif identification, in particular, identification of feedback or feedforward loops, and/or determining association with inflammation.

In a preferred embodiment of the system of the invention, transcriptomics data obtained from the subject after administration of a drug are also provided in the database. More preferably, the transcriptomics data are used to model the datasets in the database. Most preferably, datasets from the database are eliminated if no match is found between the identifier for a molecule in the dataset and the molecule identifier in the transcriptomics data.

In a preferred embodiment of the aforementioned system of the invention, said drug is a multicomponent drug. More preferably, said multicomponent drug is Traumeel.

In a preferred embodiment of the system of the invention, the visualization unit comprises a computer program-based algorithm which graphically arranges highly connected nodes close to each other and/or which identifies only nodes for graphical display that are intramodularly connected.

The present invention also relates to a method for simulating molecular interactions involved in inflammation in a subject, said method comprising
(I) providing a processing unit comprising
   (a) a database comprising a plurality of datasets each comprising
      (i) at least an identifier for a molecule suspected to be involved in the pathological process,
      (ii) data on molecular interactions of the said molecule with one or more other molecule, and
      (iii) at least one data characteristic that is allocated to the dataset, wherein each dataset has at least one relation with another dataset in the database based on molecular interactions;
      wherein the datasets are grouped into data compartments comprising datasets having identical data characteristics; and
      wherein the data characteristics are indicative for the biological function of a molecule in inflammation;
      and
   (b) an computer program-based algorithm implemented in the processing unit which generates a network map based on the plurality of datasets in the database and which allows for identifying nodes within network map based on predefined parameters;
(II) generating a network map based on the plurality of datasets in the database and identifying nodes within network map based on predefined parameters by executing the computer program-based algorithm implemented in the processing unit; and
(III) determining molecular interactions involved in inflammation in the identified nodes by using a visualization unit whereby the molecular interactions involved in inflammation in a subject are simulated.

In a preferred embodiment of the method of the invention, said inflammation comprises inflammation resolution.

In a further preferred embodiment of the method of the invention, said molecule is identified from a damage associated molecular pattern (DAMP) or from a pathogen associated molecular pattern (PAMP). More preferably, said DAMP and/or said PAMP are derived from evaluation of publications in public scientific databases using an automated evaluation algorithm which identifies molecules suspected to be involved in inflammation.

In a preferred embodiment of the method of the invention, said biological function of a molecule in inflammation is selected from the group consisting of: Mast Cell degranulation, Macrophage Differentiation, Myeloid Cell Differentiation, Lymphocyte Differentiation, Immune Cell Differentiation, Regulation of Hemopoiesis, Initiation of Innate Immune Response, Pattern recognition Receptor Signaling Pathways, Cytokine Production, Regulation of Adaptive Immune Responses, T-cell Mediated Immune Response, T cell Selection, Regulation of B cell Proliferation, Regulation of Immunoglobulin Secretion, Regulation of T cell Proliferation, Regulation of Lymphocyte Proliferation, Inflammation Resolution, Gene Expression Regulation, Protein Modification, Blood Vessel Development, Immune Response, Hemopoiesis, Neuronal Development, and Protein Transport.

In a preferred embodiment of the method of the invention, said generating a network map based on the plurality of datasets in the database and identifying nodes within network map comprises gene prioritization, determining node degree, determining betweennecess centrality, motif identification, in particular, identification of feedback or feedforward loops, and/or determining association with inflammation.

In a preferred embodiment of the method of the invention, transcriptomics data obtained from the subject after administration of a drug are also provided in the database. More preferably, the transcriptomics data are used to model the datasets in the database. Most preferably, datasets from the database are eliminated if no match is found between the identifier for a molecule in the dataset and the molecule identifier in the transcriptomics data.

In a preferred embodiment of the method of the invention, said drug is a multicomponent drug. More preferably, said multicomponent drug is Traumeel.

In yet a preferred embodiment of the method of the invention, the visualization unit comprises a computer program-based algorithm which graphically arranges highly connected nodes close to each other and/or which identifies only nodes for graphical display that are intramodularly connected.

The present invention also contemplates the use of the system of the present invention for simulating molecular interactions involved in inflammation in a subject. More preferably, said simulating molecular interactions involved in inflammation in a subject is applied for determining drug actions.

All references cited throughout this specification are herewith incorporated in their entireties as well as with respect to their disclosure content specifically mentioned.

### FIGURES

FIG. 1: User interface of the tool which we designed for data storage and the integration of functions which allow for an automated handling, e.g. for the export of the data into the molecular interaction map (MIM) system.

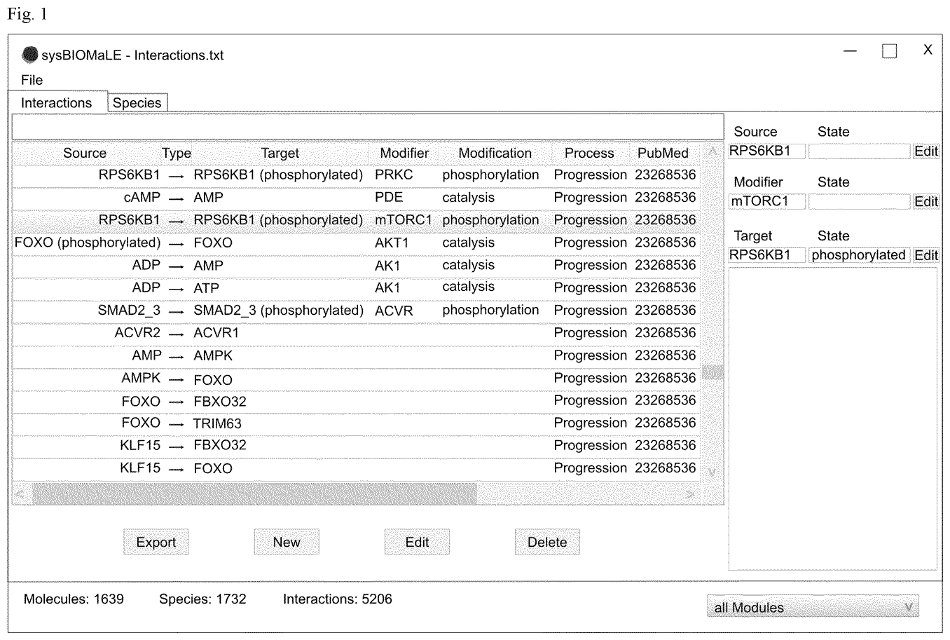

FIG. 2: Workflow for the construction of a comprehensive MIM around acute inflammation. It was started with the identification of seed molecules (left panel of the figure). Subsequently, experimentally validated interacting molecular partners from several databases and literature mining (middle) were extracted. Finally, various regulatory layers (miRNA, TFs, lncRNAs) from state-of-art databases were added. Also included were metabolites associated with inflammation resolution. Further regulatory layers such as Drug molecules may be integrated (shown on the right).

FIG. 3: Interconnectivity of the network displayed as the amount of interactions between two modules.

FIG. 4: Various steps for layouting network components and the interactions among them.

FIG. 5: Common regulatory core among all the primary clinical indications of acute inflammation MIM. TFs are shown as grey nodes while the miRNAs as orang node.

FIG. 6: Common molecules among the 5 primary clinical indications of acute inflammation. Bursitis shares 86% of the nodes with tendinitis while 40% of the total nodes are common with tenosynovitis.

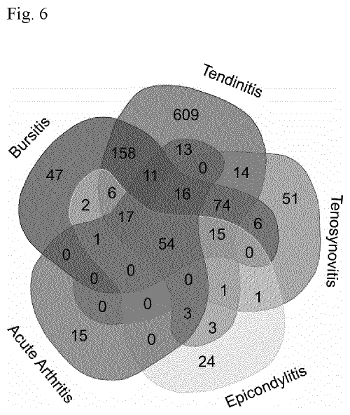

FIG. 7: Mapping of top 1000 up-regulated genes to gene/phenotype ontologies. Top 1000 genes of mouse transcriptome were mapped to inflammation related gene ontologies in a time dependent manner. Graphs show process specific number of genes within the top 1000 as the percentage of the whole GO-set.

### EXAMPLES

The following Examples are merely meant to illustrate the invention. They shall, whatsoever, not be construed as limitations for the scope.

### Example 1

### Generating of a Network Map of Molecular Interactions in Inflammation (molecular Interaction Map, MIM)

Identification of Seed Molecules for the Construction of MIM

Construction of MIM is a highly structured workflow which starts with few seed molecules that are further extended with experimentally validated molecular components and several layers of regulatory information. Seed molecules for the construction of MIM were identified in three steps:
1) Screening of damage associated molecular patterns (DAMPs)
2) Screening of pathogen associated molecular patters (PAMPs)
3) Analysis of selected acute inflammatory clinical indication networks.

DAMPs and PAMPs were identified by manual literature search of more than 50 original research papers and review articles. We mainly focused on the DAMPs that are frequently appeared in the literature on acute inflammation due to damage of muscles/connecting tissues and physical injuries (Table 1).

TABLE 1
List of DAMPs frequently identified in acute inflammation

| DAMP | Full name |
|---|---|
| HMGB1 | high-mobility group box 1 |
| HA | Glycosaminoglycan hyaluronan |
| HS | Heparan sulphate |
| UA | Uric Acid |
| IL-1α | interleukin (IL)-1α |
| IL-1β | interleukin (IL)-1β |
| IL-16 | interleukin 16 |
| IL-18 | interleukin 18 |
| FGF-2 | fibrobast growth factor |
| Gal-3 | Galectin-3 |
| Gal-1 | Galectin-1 |
| EMAP-II | endothelial monocyte-activating polypeptide-II |
| NLRP3 | NLR Family Pyrin Domain Containing 3 |
| PYCARD | PYD And CARD Domain Containing |
| MIF | Macrophage migration inhibitory factor |
| CASP1 | cysteine-aspartic acid protease 1 |
| RP S19 | Cross-linked dimer of ribosomal protein S19 |
| LPC | Lysophosphatidylcholine |
| iPLA2 | Intracellular Membrane-Associated Calcium- |
| | Independent Phospholipase A2 Beta |
| TyrRS | Tyrosyl-TRNA Synthetase |
| S100A8/ | Protein S100-A8 and S100 A-9 |
| S100A9 | |
| CRT | Calreticulin |

For the PAMPs, the literature related to the acute inflammation due to infection with various pathogens was screened. Foreign particles interact with the receptors to trigger immune responses and thus associated receptors were used as the seed molecules for the construction of MIM. List of PAMP and recognizing receptor is shown in Table 2 below:

TABLE 2
List of PAMPs and their recognizing
receptors in acute inflammation

| PAMP | Recognizing receptor |
|---|---|
| Cytoplasmic DNA | AIM2 |
| Bacterial Flagelli | NLRC4 |
| Bacterial Type III Secretion Systems | NLRC4 |
| Bacillus Anthracis Lethal Toxin | NLRP1 |
| ATP | NLRP3 |
| Marine toxin maitotoxin | NLRP3 |
| Crystals (Urate, Calcium | NLRP3 |
| Pyrophosphate Dihydrate, | |
| Silica, Asbestos) | |
| Diaminopimelic Acid | NOD1 |
| Muramyl Dipeptide | NOD2 |
| Triacyl Lipopeptides | TLR1, TLR2 |
| Peptidoglycan | TLR2 |
| Lipoarabinomannan | TLR2 |
| Envelope Glycoproteins | TLR2 |
| Phospholipomannan | TLR2 |
| Porins | TLR2 |
| Diacyl Lipopeptides | TLR2, TLR6 |
| Lipoteichoic acid | TLR2, TLR6 |
| dsRNA | TLR3, RIG1, DDX58, IFIH1, |
| | EIF2AK2 |
| Lipopolysaccharide | TLR4 |
| Mannan | TLR4 |
| Flagellin | TLR5 |
| ssRNA | TLR7, TLR8 |

To keep the MIM close to clinical relevance, the literature mining (>50 publications and >15 review articles) on molecular events associated with various acute inflammatory clinical indications was also performed. From a list of 29 primary clinical indications of acute inflammations provided by HEEL GmbH, publically available literature (PubMed, OMIM) and disease-gene association database (DisGeNET, DISEASE, KEGG disease) was screened to find the genes associated with the clinical indication. Most of these databases are based on text mining algorithms to predict the relationship between biological entities, which in many cases results in false positive information. The association of key genes with the clinical indications was cross checked manually by screening the associated publication. The key genes associated with each of the primary clinical indications of acute inflammation are summarized in Table 3.

TABLE 3
List of primary clinical indications of acute inflammation
with associated key genes, number of nodes and interaction
in molecular interaction network

| Name of Clinical | | |
|---|---|---|
| indication | UMLS Id | Key genes |
| Bursitis | C0006444 | IL1B, IL6, COX-2, |
| | | CXCL12 |
| Subacromial Bursitis | C0546953 | IL1B, IL6, CXCL12, |
| | | VEGFA |
| Olecranon Bursitis | C0263962 | KDR |
| Tendinitis | C0039503 | TP53, SIRT1 |
| Tenosynovitis | C0039520 | HLA-B, HLA-C, TNF |
| Epicondylitis | C0039516 | PRG4, COL5A1 |
| De Quervain disease | C0149870 | ESR2 |
| Acute Arthritis | C0263678 | GPI, RPS19 |

A unique set of genes from Table 1-3 was finally created as the seed molecules around which a comprehensive MIM was constructed. In total, 53 seed molecules around which the MIM was created were used.

Construction of MIM from Seed Molecules (Undirected Graph)

For all the seed molecules, information about the molecular interactions in the scope of acute inflammation was collected manually from the literature and also from several automated tools such as BisoGenet app available on Cytoscape4.0 which connects large number of databases.

In particular, collected was information about:
source and target molecules
possible modifiers (e.g. enzymes)
type of modification (e.g. catalysis, phosphorylation)
state of the molecules (e.g. phosphorylated)
type of the molecules (proteins, miRNA, complexes, DNA, simple molecules)
type of the interaction (positive, negative)
references as PubMed-IDs

To structure the information in one place and bring together the data generated from other sources (e.g. STRNG-Database), a tool was created, which allows easily storage, handling and alteration of the Data and gives an opportunity to implement new function to manipulate the Data in respect of the upcoming tasks. The tool was created in the C# Microsoft .NET Framework using Microsoft Visual Studio Community 2017 Version 15.9.0. Fehler! Verweisquelle konnte nicht gefunden werden. shows the user interface in the current state of development (December 2018).

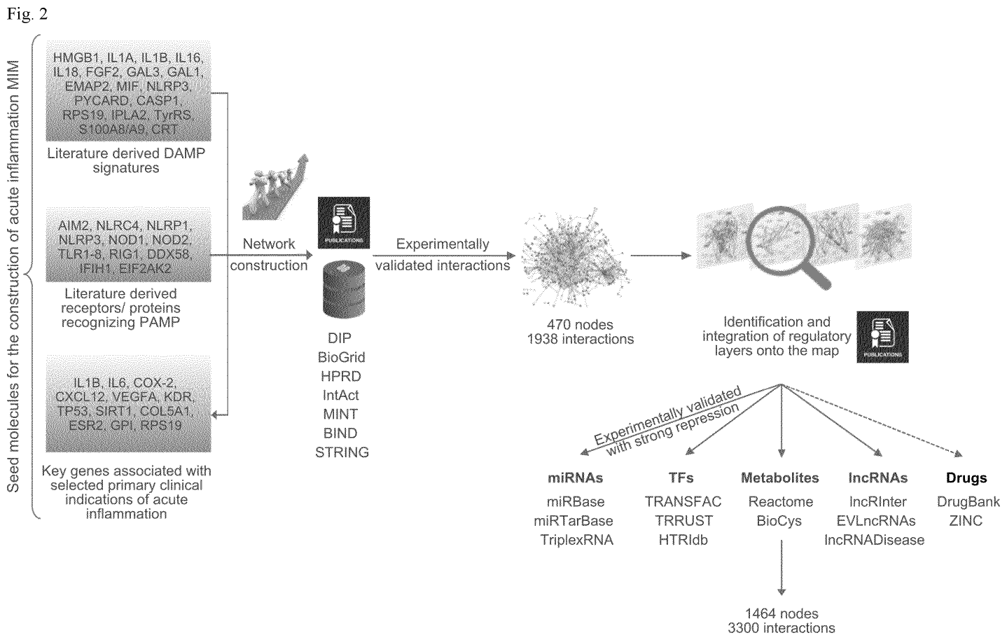

For this, all the seed molecules and extracted information about the first experimentally validated molecular targets along with direct interactions among themselves if any were provided. Mainly considered were experimentally validated molecular targets from DIP, BioGrid, HPRD, IntAct, MINT, BIND and String databases. Furthermore, several experimentally validated regulatory layers were connected, which include miRNAs from mirBase, miRTarBase, TriplexRNA; transcription factors from TRNSFAC, TRRUST and HTRIdb; lnc RNAs from lncRInter, EVLncRNAs, lncRNADisease databases. It is already evident that fatty acid metabolism play an important role in the biosynthesis of specialized pro-resolving molecules (including resolvins, protectins and maresins). Also collected was information about the biosynthesis of these inflammation resolution mediators from Reactome database and published literature and included them in the MIM along with all the associated enzymes. The overall process is summarized in FIG. 2. Currently the cytoscape version of the network has 1464 nodes and 3300 interactions.

Annotation and Enrichment of the MIM

Every gene in the MIM was enriched by its specific UNIPROT-, HGNC-, RefSeq-, Ensemble-, PubMed- and NCBI-ID as well as common aliases and the full name of the encoding Protein. This information was collected as a single file downloaded from the HGNC database on Nov. 14, 2018. In case of simple molecules, the CHEBI-ID was fetched for each molecule separately from the CHEBI database while gathering the interaction data from the literature.

Modularization of the MIM Based on Acute Inflammation Related Processes

For easy navigation and visualization of the MIM, the MIM was divided into various functional modules by assigning the molecules to gene ontology terms focused on acute inflammation. For this, the CytoScape plugin ClueGO was used, which utilizes the GeneOntology (GO) database to structure a list of genes into modules of GO-Terms. The results can be adjusted manually by defining parameters, e.g. the range of tree levels of the term in the GO-Hierarchy, the p-value for the association or the amount of genes per modules. To focus on immunology related processes, the GO database “immunological_process” was used in the first run of modularization and 230 of all 1464 (FIG. 2) genes to 116 GO terms were assigned, which were merged manually into 17 modules. In the second run, the more general database “biological_process” was used and 778 of the remaining 1234 genes to 79 GO terms were assigned, which again were merged into seven modules.

Inflammation Resolution Module

The major goal to design and construct the MIM was to understand the acute inflammation and inflammation resolution processes. As inflammation resolution process is not included in the GeneOntology database, genes were assigned manually to this module based on information available in the literature. A large number of specialized pro-resolving mediators (SPMs) were found for which the information on the biosynthesis and signaling pathways was extracted from the literature (PubMed) and pathway database (Reactome). Non-enzymatic mediators are named systematically with a nomenclature that unifies synonyms used in different publications. The nomenclature is based mainly on the ChEBI name (https://www.ebi.ac.uk/chebi/). Enzymes and proteins are named with their respective UniProt gene names (https://www.uniprot.org/). The value/direction of an interaction is characterized as positive, negative or neutral (with further specification, if necessary; e.g. “neutral (translocation from neutrophils to platelets)”), and the type of interaction is given in appropriate terminology (e.g. “catalysis”, “inactivation”, “phosphorylation”, etc.). If the interaction is a biosynthesis or inactivation step, the product of the reaction is further characterized as either a metabolite or a mediator. Additionally, information about cell types associated with the interaction is added if available. Lastly, every interaction is referenced by the PMID of the publications used in the acquisition of information about it. So far, this module contains 256 interactions, including (not limited to) the biosynthesis pathways of protectins, maresins, resolvins, lipoxins, respective precursors, respective inactivation pathways, and associated receptors. Fehler! Verweisquelle konnte nicht gefunden werden. shows all 25 modules with their total number of genes in which a gene can occur in several modules.

TABLE 4
List of the generated modules with their
respective number of associated genes

| Module Term | #Genes |
|---|---|
| Mast Cell degranulation | 17 |
| Macrophage Differentiation | 16 |
| Myeloid Cell Differentiation | 73 |
| Lymphocyte Differentiation | 65 |
| Immune Cell Differentiation | 61 |
| Regulation of Hemopoiesis | 51 |
| Initiation of Innate Immune Response | 111 |
| Pattern recognition Receptor Signaling Pathways | 56 |
| Cytokine Production | 27 |
| Regulation of Adaptive Immune Responses | 35 |
| T-cell Mediated Immune Response | 23 |
| T cell Selection | 18 |
| Regulation of B cell Proliferation | 20 |
| Regulation of Immunoglobulin Secretion | 10 |
| Regulation of T cell Proliferation | 75 |
| Regulation of Lymphocyte Proliferation | 42 |
| Inflammation Resolution | 230 |
| Gene Expression Regulation | 246 |
| Protein Modification | 193 |
| Blood Vessel Development | 123 |
| Immune Response | 146 |
| Hemopoiesis | 118 |
| Neuronal Development | 164 |
| Protein Transport | 168 |

Furthermore, the biosynthetic pathways of various inflammation mediators (leukotrienes, eoxins, prostaglandins, thromboxanes, hepoxilins and trioxilins) along with their precursors and respective inactivation pathways were also included in the MIM as an inflammation mediator module.

Network Visualization

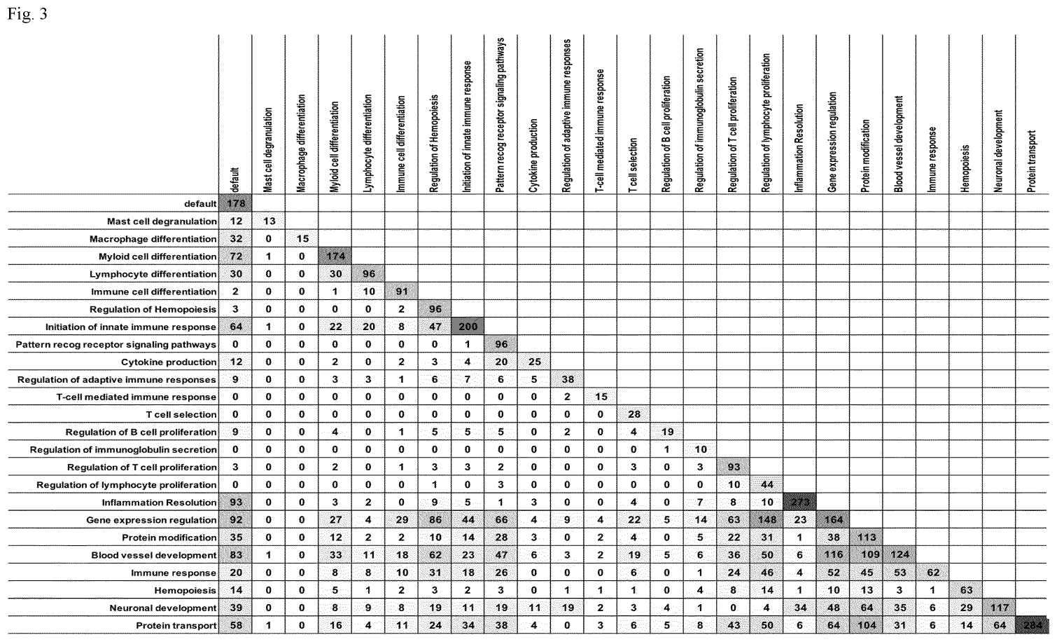

To visualize the molecular interactions in a molecular interaction map (MIM), they need to be designed in applications capable to create and display this information in the systems biology graphical notation (SBGN). Most of these applications furthermore allow the visualization and mapping of experimental data in the map. Because of the big size of the MIM, a manual creation is either not possible, or require considerable amount of time and manpower. To overcome this, an automated export function was developed from the dataset into the Systems Biology Markup Language (SBML) file format which describes the MIM in the SBGN style and is readable by the application CellDesigner™ that was used as a tool to display and layout the map as well as to integrate time series transcriptomic data. The function automatically places all molecules in their respective modules in the map and applies shape and color to the node depending on the molecule type and also draws the interactions. The interconnectivity of the modules is visualized in FIG. 3 below indicating the number of interactions between two of the modules.

Layout of MIM for Easy Navigation

Molecular Interaction Maps (MIMs) MIMs are important source of molecular mechanisms of diseases, which can be used to formulate new hypotheses that can subsequently be tested by experiments. Such maps are enormous in size (i.e. contain large number of components) and complexity (i.e. containing intricate recurring structures), which make them difficult to visualize and explore. Better visualization will facilitate understanding the underlying mechanisms and enable efficient sharing and usage of knowledge in biomedical research. Although, the maps construction and visualization tools like CellDesigner and Cytoscape provide built-in layouting options but for large and modularized MIMs they cannot produce efficient visualization. The automatic layouting algorithms work well only for small maps. Visualization of large MIMs requires enormous manual efforts. Towards this, step by step procedures were developed to arrange network components and interactions for better visualization.

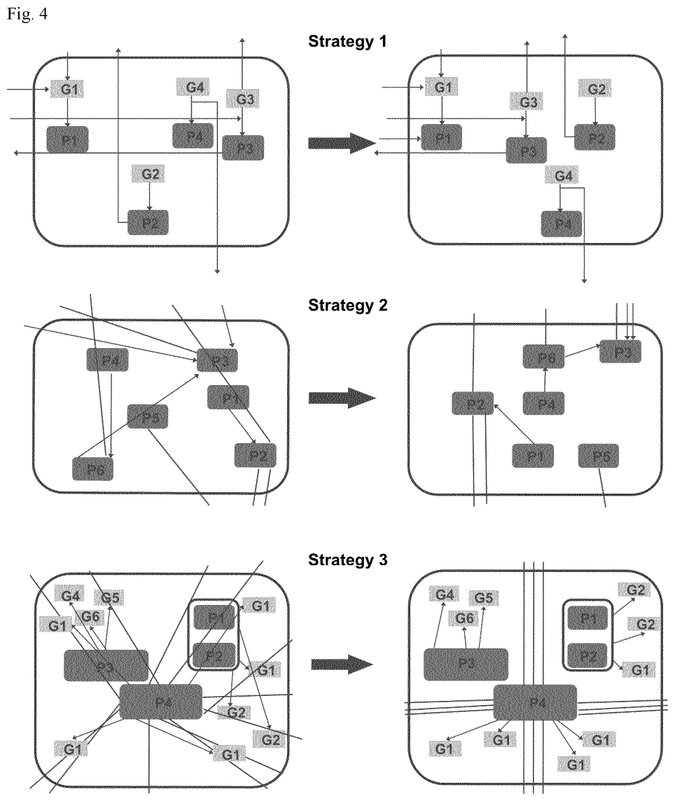

Step 1: Components were organized in a module based on their interactions with other modules (i.e. inter modular connections) in a way that most of the interacting arrows shouldn't cross each other. For example, the FIG. 4a on the left shows the connection of genes (G1, . . . , 4) and proteins (P1, . . . , 4) with other modules of the map.

In this case:
G1, G3 and P3 are interacting with the left part of the network.
G1, G2, P2 are interacting with the upper part of the network.
G4 and P4 are interacting with the lower part of the network.

Here, based on the position of inter modular interactions, the components were placed either to the left, upper or bottom part of the module.

Step 2: The network components are organized based on their interactions within a module as well as their interactions with other modules of the map as shown in the FIG. 4b.

Step 3: In this step, other readability issues were resolve for components such as proteins and complexes that have many inter- and intra-modular connections (FIG. 4c).

Zoomable Image of the MIM in Browser

An aim was to bring the MIM in browser so that the community around the acute inflammation can easily be connected. First the literature was surveyed for algorithms and technique for displaying the MIM as an interactive image. OpenLayer and Google Maps based techniques were selected to bring the MIM in browser for easy visualization. More specifically, the MIM was divided into three layers. Basic layers are modules in the top level, sub-modules and species in the middle layer and species and interactions in the bottom layer. To reduce the computational effort and the requirements to the local internet tiling of the image was carried out. Tiling is a technique to cut images into a matrix of smaller images of the same size and store it in a specific folder structure. With this, only the currently viewed parts of the MIM can be loaded and displayed. Python scripts for tiling and layering were applied to test if the CellDesigner export of our map fits to the postproduction process of the MIM. MINERVA [https://minerva.pages.uni.lu] platform was used to visualize the map. A local MINERVA instance was installed and tested with MIM for various security and reliability issues.

MINERVA provides the possibility to use OpenLayer as the basic visualization library to enhance the data security in comparison to GoogleMaps. The tiling and layering process is fully automated and basic mouse operations for zooming and panning the MIM are also supported. In addition, the user can select nodes to see the underlying annotations and at the same time connect with various drug and chemical databases directly from MINERVA interface for further network analyses.

Making the MIM as a Directed Graph

Providing direction (e.g. activation, inhibition) to the edges connecting various nodes in the network is a crucial step for analyses and prediction of biomarkers and therapeutic candidates. This is one of the most important steps in the construction of a reliable disease map and requires lots of manual efforts. All the associated publications that highlight the interactions among biological partners were cross checked in order to find the regulation type and annotate the reactions accordingly. Systems biology resources such as BioModel databases were also used to assign regulatory directions to the connected components using in house scripts. Many of the databases are using text mining approaches to even highlight experimentally validated interactions with false positive information. This was a time taking exercise to define the regulation type (e.g. activation, suppression, activated complex etc.) in the construction of a disease MIM. However, this exercise is worth doing to properly curate the MIM and also for the downstream analysis related to the identification of regulatory motifs and prioritization of network components. So far, around 70% of the total interactions in the network are directed.

Identification of Common Regulators from Selected Primary Clinical Indications of Acute Inflammation

Some of the primary clinical indications of acute inflammation were identified, where the information about key genes was available in the literature. For those clinical indications, the common molecular regulators should be found. To achieve this task, first the clinical indication specific MIM was constructed using the workflow described in FIG. 2. For the miRNA regulatory layer, we used the miRTarBase release 6.1 and only considered those miRNAs which are experimentally shown to have strong repression efficiency on the target genes. List of key genes, number of nodes, interactions, number of known transcription factors and number of miRNAs strongly regulating target genes in the networks are shown in Table 5.

TABLE 5
Summary of MIM constructed around the selected primary
clinical indication of acute inflammation

| Name of Clinical | No. of | No. of | No. of | No. of |
|---|---|---|---|---|
| indication | nodes | interactions | TFs | miRNAs |
| Bursitis | 407 | 1013 | 158 | 217 |
| Subacromial | 407 | 1071 | 145 | 218 |
| Bursitis | | | | |
| Olecranon | 341 | 891 | 103 | 192 |
| Bursitis | | | | |
| Tendinitis | 994 | 4617 | 404 | 308 |
| Tenosynovitis | 232 | 423 | 61 | 132 |
| Epicondylitis | 127 | 188 | 18 | 89 |
| De Quervain | 300 | 677 | 95 | 176 |
| disease | | | | |
| Acute Arthritis | 130 | 171 | 26 | 94 |

To identify common genes, TFs and miRNAs shared by all the primary clinical indications studied here, Cytoscape merge network tool was used to find the interactions among the clinical indication-specific networks. Transcription factors STAT3 and SP1 were found along with 48 experimentally validated miRNAs as the common regulators in all the 8 primary clinical indications of acute inflammation for which MIM was constructed (FIG. 4).

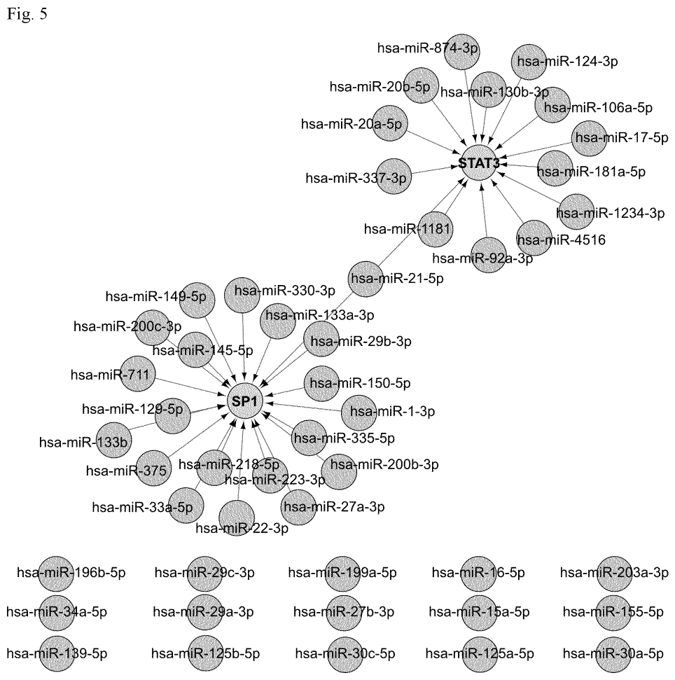

In case of bursitis, tendinitis, tenosynovitis, epicondylitis and acute arthritis, there were 54 common candidates (FIG. 5) including 4 transcription factors (EGR1, STAT3, SP1 and JUN) and 50 miRNAs. Role of STAT3 through IL-6 driven signaling is previously reported in the termination of inflammatory recruitment of neutrophils which is a crucial checkpoint in inflammation resolution (PMID: 18641358). Among the common miRNAs, we found miR-155 which has been identified as central regulator of the immune system (PMID: 19596814). Among others, miR-21 and miR-203a are already known for their role in acute inflammation resolution (PMID: 20956612).

### Example 2

### Integration of Traumeel Transcriptomics Data from Mouse Wound Healing Model onto the MIM and Investigations of the Traumeel Induced Phenotype

Integration of Traumeel Transcriptomics Data from Mouse Wound Healing Model onto the MIM

The RNA-seq data provided by HEEL encompassed 55419 transcripts also partly encoding for gene isomers. Gene expression levels were quantified by measurement of RPKM (reads per kilo million) values for each transcript. RPKM values were measured for 6-7 samples (n=6/7), at 8 time points (0 h-192 h), and for 4 conditions (untreated, saline-treated, Traumeel injection alone, Traumeel injection plus ointment). It was realized that in the RNA-seq data, RPKM values were provided for each of the transcripts associated with a particular gene. Further in the process of identification and analysis of differentially expressed genes and their association with higher level processes were identified by mapping GO terms. So far the association of GO terms at the gene transcript level is largely missing and generally the same GO terms are mapped to all the gene transcripts. In order to quickly estimate the differentially expressed genes from the RNA-seq data in different conditions and to map them on the MIM, the RPKM values were added of all the isomers of a gene for each replicate separately followed by calculating their average in different conditions. Then calculated log(2)-fold change of each of the gene was calculated by comparing various conditions (untreated vs traumeel injection alone; saline vs traumeel injection alone; untreated vs traumeel gel; and saline vs traumeel gel). Further, the log(2)-fold change data was integrated onto the MIM for each of the time points.

From the cytoscape version of the MIM, the differentially expressed network components (>1.5 log(2)-fold change and p-value<0.05) were extracted in untreated vs traumeel injection experimental condition at 12 hrs. The change in the expression pattern of this differentially expressed network was investigated at higher time point. It was observed that after 120 hrs, many of the nodes has same expression profile between the above mentioned two experimental conditions. At 192 hrs, none of the node differentially expressed indicating that inflammation is already resolved at this time point.

Evaluating the Influence of Traumeel Treatment on Biological Processes and Phenotypes Based on log(2)-Fold Change in Gene Expression

In a first attempt to identify how Traumeel affects core regulatory processes in the mouse wound healing model, gene sets were identified for selected biological processes and phenotypes using the Gene Ontology Consortium (GO, http://www.geneontology.org) and the Mouse Phenome Database (MP, https://phenome.jax.org). The pre-processed time course data (averaging gene isoforms and samples) for each GO/MP gene set was then extracted from the transcriptomic data and visualized in a line plot.

To get an impression on how the selected biological processes and phenotypes behave as a whole (shifting the focus from single molecules to complex functions) mean expression values for each time point were calculated and visualized as log(2)-fold change. The blue line shows the differential expression of the whole gene set whereas the orange respectively the grey line represent the gene sets for saline (S) and Traumeel treatment (T) alone. Saline was used as a reference in this context due to the assumption that the injection procedure itself as well as carrier solutions, e.g. saline, might influence transcription already.

Evaluating the Influence of Traumeel Treatment on Biological Processes and Phenotypes Based on Top 1000 Up-Regulated Genes

In order to identify the biological processes mostly affected by Traumeel treatment we extracted the top 1000 up-regulated genes for each time point (12 h-192 h) in untreated (U) and Traumeel treated (T) animals and mapped them to gene ontologies with the EnrichR gene set enrichment analysis web server (http://amp.pharm.mssm.edu/Enrichr/). In summary, a list of 3624 GO-terms was generated and submitted for further evaluation. As a first selection we extracted all T cell-, neutrophil-, macrophage- & migration-related gene ontologies. Secondly, we chose two biological processes related to inflammation in general for exemplification, namely “Neutrophil migration” and “Neutrophil degranulation”.

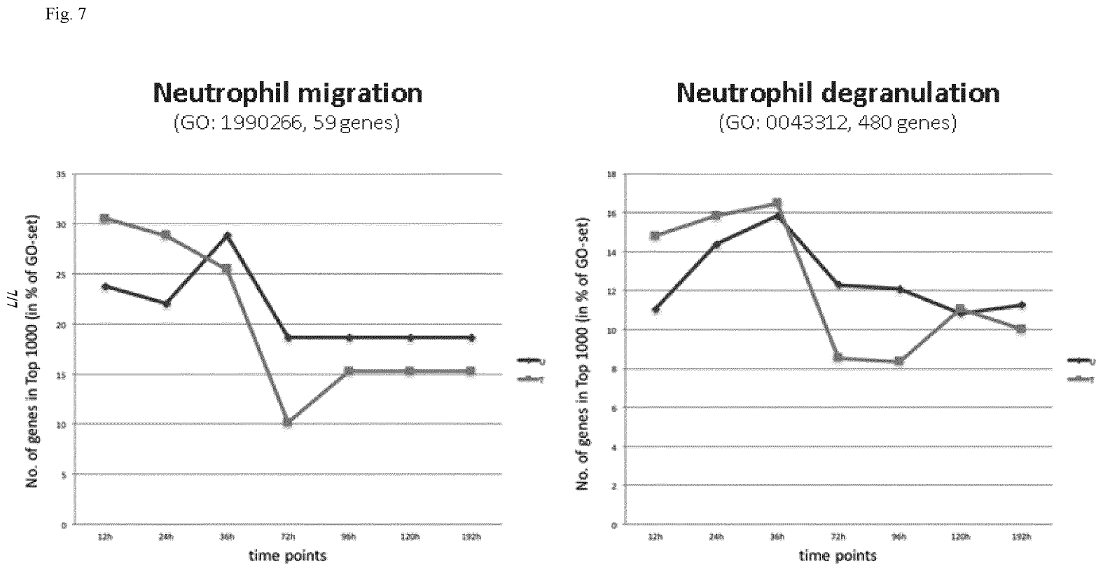

FIG. 7 shows the number of genes present in the top 1000 at each time point as percentage of the whole GO-related gene set, with the x-axis showing the time points and the y-axis the number of GO-related genes in the top 1000 in percent of the whole GO-set. As an example: If a GO-term contains 480 genes and 48 of those are present in the Top 1000 at t=24 h the y-value for this time point will be 10%. The comparison of Traumeel treated animals with untreated animals (FIG. 7) allows two major conclusions: First, the overall trend in genes activated in percent of GO-set remains similar (i.e. no disruption of physiology). Second, Traumeel seems to stimulate the expression of genes involved in inflammation related biological functions in the beginning (stronger initiation) whereas it inhibits the GO-set involvement towards the end (stronger resolution). This directed process intensification might be accountable for the inflammation resolving properties of Traumeel.

### CITED LITERATURE

Dreyer et al. 2018, BBA—Molecular Basis of Disease 1864: 2315-2328;

Khan et al. 2018, nature communications 8: 198;

Netea et al. 2017, Nature Immunol. 18(8): 826-831;

Sadeghi et al. 2016, PLOS one, DOI:10.1371/journal.pone.0168760;

Steffen et al. 2017, Journal of Biotechnology 261: 85-96;

Wolkenhauer 2002, BioSystems 65: 1-18

1. A system for simulating molecular interactions involved in inflammation in a subject, said system comprising
(I) a processing unit comprising
(a) a database comprising a plurality of datasets each comprising
(i) at least an identifier for a molecule suspected to be involved in the pathological process,
(ii) data on molecular interactions of the said molecule with one or more other molecule, and
(iii) at least one data characteristic that is allocated to the dataset,
wherein each dataset has at least one relation with another dataset in the database based on molecular interactions;
wherein the datasets are grouped into data compartments comprising datasets having identical data characteristics; and
wherein the data characteristics are indicative for the biological function of a molecule in inflammation;
and
(b) an computer program-based algorithm implemented in the processing unit which generates a network map based on the plurality of datasets in the database and which allows for identifying nodes within network map based on predefined parameters; and
(II) a visualization unit which allows for determination of the molecular interactions involved in inflammation in the identified nodes.

2. The system of claim 1, wherein said inflammation comprises inflammation resolution.

3. The system of claim 1, wherein said molecule is identified from a damage associated molecular pattern (DAMP) or from a pathogen associated molecular pattern (PAMP).

4. The system of claim 3, wherein said DAMP and/or said PAMP are derived from evaluation of publications in public scientific databases using an automated evaluation algorithm which identifies molecules suspected to be involved in inflammation.

5. The system of claim 1, wherein said biological function of a molecule in inflammation is selected from the group consisting of: Mast Cell degranulation, Macrophage Differentiation, Myeloid Cell Differentiation, Lymphocyte Differentiation, Immune Cell Differentiation, Regulation of Hemopoiesis, Initiation of Innate Immune Response, Pattern recognition Receptor Signaling Pathways, Cytokine Production, Regulation of Adaptive Immune Responses, T-cell Mediated Immune Response, T cell Selection, Regulation of B cell Proliferation, Regulation of Immunoglobulin Secretion, Regulation of T cell Proliferation, Regulation of Lymphocyte Proliferation, Inflammation Resolution, Gene Expression Regulation, Protein Modification, Blood Vessel Development, Immune Response, Hemopoiesis, Neuronal Development, and Protein Transport.

6. The system of claim 1, wherein said computer program-based algorithm implemented in the processing unit which generates a network map based on the plurality of datasets in the database and which allows for identifying nodes within network map comprises rules for gene prioritization, determining node degree, determining betweennecess centrality, motif identification, in particular, identification of feedback or feedforward loops, and/or determining association with inflammation.

7. The system of claim 1, wherein transcriptomics data obtained from the subject after administration of a drug are also provided in the database.

8. The system of claim 7, wherein the transcriptomics data are used to model the datasets in the database.

9. The system of claim 8, wherein datasets from the database are eliminated if no match is found between the identifier for a molecule in the dataset and the molecule identifier in the transcriptomics data.

10. The system of claim 7, wherein said drug is a multicomponent drug.

11. The system of claim 10, wherein said multicomponent drug is Traumeel.

12. The system of claim 1, wherein the visualization unit comprises a computer program-based algorithm which graphically arranges highly connected nodes close to each other and/or which identifies only nodes for graphical display that are intramodularly connected.

13. A method for simulating molecular interactions involved in inflammation in a subject, said method comprising
(I) providing a processing unit comprising
(a) a database comprising a plurality of datasets each comprising
(i) at least an identifier for a molecule suspected to be involved in the pathological process,
(ii) data on molecular interactions of the said molecule with one or more other molecule, and
(iii) at least one data characteristic that is allocated to the dataset,
wherein each dataset has at least one relation with another dataset in the database based on molecular interactions;
wherein the datasets are grouped into data compartments comprising datasets having identical data characteristics; and
wherein the data characteristics are indicative for the biological function of a molecule in inflammation;
and
(b) an computer program-based algorithm implemented in the processing unit which generates a network map based on the plurality of datasets in the database and which allows for identifying nodes within network map based on predefined parameters;
(II) generating a network map based on the plurality of datasets in the database and identifying nodes within network map based on predefined parameters by executing the computer program-based algorithm implemented in the processing unit; and
(III) determining molecular interactions involved in inflammation in the identified nodes by using a visualization unit whereby the molecular interactions involved in inflammation in a subject are simulated.

14. The method of claim 13, wherein said inflammation comprises inflammation resolution.

15. The method of claim 13, wherein said molecule is identified from a damage associated molecular pattern (DAMP) or from a pathogen associated molecular pattern (PAMP).

16. The method of claim 15, wherein said DAMP and/or said PAMP are derived from evaluation of publications in public scientific databases using an automated evaluation algorithm which identifies molecules suspected to be involved in inflammation.

17. The method of claim 13, wherein said biological function of a molecule in inflammation is selected from the group consisting of: Mast Cell degranulation, Macrophage Differentiation, Myeloid Cell Differentiation, Lymphocyte Differentiation, Immune Cell Differentiation, Regulation of Hemopoiesis, Initiation of Innate Immune Response, Pattern recognition Receptor Signaling Pathways, Cytokine Production, Regulation of Adaptive Immune Responses, T-cell Mediated Immune Response, T cell Selection, Regulation of B cell Proliferation, Regulation of Immunoglobulin Secretion, Regulation of T cell Proliferation, Regulation of Lymphocyte Proliferation, Inflammation Resolution, Gene Expression Regulation, Protein Modification, Blood Vessel Development, Immune Response, Hemopoiesis, Neuronal Development, and Protein Transport.

18. The method of claim 13, wherein said generating a network map based on the plurality of datasets in the database and identifying nodes within network map comprises gene prioritization, determining node degree, determining betweennecess centrality, motif identification, in particular, identification of feedback or feedforward loops, and/or determining association with inflammation.

19. The method of claim 13, wherein transcriptomics data obtained from the subject after administration of a drug are also provided in the database.

20. The method of claim 19, wherein the transcriptomics data are used to model the datasets in the database.

21. The method of claim 20, wherein datasets from the database are eliminated if no match is found between the identifier for a molecule in the dataset and the molecule identifier in the transcriptomics data.

22. The method of claim 19, wherein said drug is a multicomponent drug.

23. The method of claim 22, wherein said multicomponent drug is Traumeel.

24. The method of claim 13, wherein the visualization unit comprises a computer program-based algorithm which graphically arranges highly connected nodes close to each other and/or which identifies only nodes for graphical display that are intramodularly connected.

25. A method of using the system of claim 1, for simulating molecular interactions involved in inflammation in a subject.

26. The method of claim 25, wherein simulating molecular interactions involved in inflammation in a subject is applied for determining drug actions.
